(19) United States
(12) Patent Application Publication
(10) Pub. No.: US 2023/0414450 A1
(43) Pub. Date: Dec. 28, 2023

(54) BLOOD TRANSFER SHIELD AND METHODS OF USE

(71) Applicant: The General Hospital Corporation, Boston, MA (US)

(72) Inventors: Ryan W. Carroll, Boston, MA (US); Kimberly Whalen, Boston, MA (US); Matthew Beatty, Malden, MA (US)

(21) Appl. No.: 18/251,878

(22) PCT Filed: Nov. 5, 2021
(86) PCT No.: PCT/US2021/058335

§ 371 Date: May 4, 2023

Related U.S. Application Data
(60) Provisional application No. 63/110,300, filed on Nov. 5, 2020.

(51) Int. Cl. A61J 1/20; A61M 39/10

(52) U.S. Cl. CPC A61J 1/20; A61M 39/10; A61M 2039/1088; A61M 2039/1083; A61M 2039/009

(57) ABSTRACT

Systems and methods for shielded fluid transfer are provided. A fluid transfer shield is provided to facilitate transferring fluid from a first container to a secondary container. The fluid transfer shield includes a shield having an exterior and defining an interior space, a first port extending from the exterior of the shield, the first port configured to be fluidly coupled to the first container, and a second port extending opposite the first port and into the interior space of the shield so that the shield laterally surrounds the second port, the second port configured to be fluidly coupled to the secondary container. A passageway is provided that extends between the first port and the second port to fluidly couple the first container and the secondary container when the first container is fluidly coupled to the first port and the secondary container is fluidly coupled to the second port.

### CROSS-REFERENCES TO RELATED APPLICATIONS

The present application is based on, claims priority to and incorporates herein by reference in its entirety U.S. Provisional Patent Application No. 63/110,300, filed Nov. 5, 2020, and entitled “Blood Transfer Shield.”

### STATEMENT REGARDING FEDERALLY SPONSORED RESEARCH

Not Applicable.

### BACKGROUND

The disclosure relates generally to controlled fluid transfer, and more specifically, to systems and methods for blood transfer from a syringe to a secondary container.

Certain fluid transfer processes, such as blood transfer within the context of phlebotomy, for example, can put users, such as healthcare or lab workers, at risk of unwanted blood or needle exposure. In particular, in certain healthcare settings, a healthcare worker may need to transfer blood from a syringe to another receptacle or secondary container. Unwanted blood exposure can occur during the transfer process of the blood to the secondary container, which may not be carried out in a closed transfer system.

### BRIEF SUMMARY

The present disclosure provides systems and methods for shielded blood transfer. In one configuration, a fluid transfer shield is provided that facilitates transferring fluid from a first container to a secondary container. The fluid transfer shield can include a shield having an exterior and defining an interior space. A first port can extend from the exterior of the shield and can be configured to be fluidly coupled to the first container. A second port can extend opposite the first port and into the interior space of the shield so that the shield laterally surrounds the second port. The second port can be configured to be fluidly coupled to the secondary container. A passageway can extend between the first port and the second port to fluidly couple the first container and the secondary container when the first container is fluidly coupled to the first port and the secondary container is fluidly coupled to the second port.

Some configurations provide a fluid transfer shield that includes a second port having a relief path to allow air flow between a secondary container and the environment when the secondary container is secured to the second port.

Some configurations provide a fluid transfer shield that includes a relief path formed by a pair of planar faces on opposing sides of a second port. The pair of planar faces can be configured to form a channel between an exterior surface of the second port and an interior surface of a secondary container when the secondary container is secured to the second port.

Some configurations provide a fluid transfer shield that includes a flange extending radially from an exterior of a shield proximate to a base of the shield.

Some configurations provide a fluid transfer shield and first and second support ribs that extend from an exterior of a shield to a flange to rigidly support the flange.

Some configurations provide a fluid transfer shield having a perimeter of a fluid transfer shield, the perimeter including a shield and a flange adjacent to a base of the shield. The perimeter can form an oblong footprint.

Some configurations provide a fluid transfer shield that includes a second port. The second port can include an interior surface that defines a passageway and an exterior surface. The exterior surface can have a tapered geometry so that a distal end of the second port is narrower than a proximal end of the second port.

Some configurations provide a fluid transfer shield that includes a first port. The first port can include a female Luer lock connector. A first container can be configured as a syringe having a male Luer lock connector to fluidly couple the syringe to the first port via the female Luer lock connector.

Some configurations provide a fluid transfer shield with a second port. The second port can extend between approximately 7 millimeters and 11 millimeters into an interior space of a shield and the shield can define a body height that is between approximately 16 millimeters and 24 millimeters.

Some configurations provide a fluid transfer shield having an opening diameter of a shield between approximately 14 millimeters and 22 millimeters and an overall height of the fluid transfer shield between approximately 22 millimeters and 33 millimeters.

Configurations of the present disclosure provide a blood transfer shield for fluidly coupling a syringe and a secondary container. The blood transfer shield can include a shield, a passageway, and a first port. The shield can have a cylindrical body and a base that defines an opening at a first end of the cylindrical body. A passageway can extend through the cylindrical body of the shield. The passageway can be formed at a second end of the cylindrical body opposite the base. The first port can form a first end of the passageway. The first port can be configured to securely engage the syringe to fluidly couple the syringe to the passageway.

In some configurations, a blood transfer shield can include a second port that forms a second end of a passageway. The second port can be laterally surrounded by a shield.

In some configurations, a blood transfer shield can include a second port that defines an exterior surface opposite of a passageway. The exterior surface can be configured to be inserted into an opening of a secondary container to fluidly couple the secondary container with the passageway.

In some configurations, a blood transfer shield can include an exterior surface having a first pair of faces and a second pair of faces. The first pair of faces can be configured as curved surfaces that extend along a second port in an axial direction and the second pair of faces can be configured as flat surfaces that extend along the second port in the axial direction.

In some configurations, a blood transfer shield can include a second pair of faces that are configured to form an air relief channel with an opening of a secondary container when the secondary container is fluidly coupled with a passageway.

In some configurations, a blood transfer shield can include a flange extending radially from a cylindrical body of a shield adjacent to an opening. First and second buttresses can extend downward from a second end of the cylindrical body to the flange.

In some configurations, a blood transfer shield can include a first port and second port that are tapered so that a respective distal end is narrower than a respective proximal end of the first and second ports.

Some configurations of the present disclosure provide a method of transferring blood from a first container to a secondary container. The method can include drawing blood into the first container, fluidly coupling the first container to a first port of a blood transfer shield, the first port extending from an exterior surface of the blood transfer shield, fluidly coupling the secondary container to a second port of the blood transfer shield, the second port extending within an interior space defined by a lateral side wall of a shield of the blood transfer shield, and expelling blood from the first container into the second container via a passageway extending between the first port and the second port.

In some configurations, a method of transferring blood from a first container to a secondary container can include fluidly coupling the first container to the second container by twisting the first container relative to a blood transfer shield.

### BRIEF DESCRIPTION OF DRAWINGS

The disclosure will be better understood and features, aspects, and advantages other than those set forth above will become apparent when consideration is given to the following detailed description thereof. Such detailed description makes reference to the following drawings which may not be drawn to scale.

FIG. 1 is an isometric view of a fluid transfer shield according to the present disclosure.

FIG. 2 is a cross-sectional isometric view of the fluid transfer shield taken along line 2-2 of FIG. 1.

FIG. 3 is a cross-sectional isometric view of the fluid transfer shield taken along line 3-3 of FIG. 1.

FIG. 4 is a cross-sectional side view of the fluid transfer shield taken along line 3-3 of FIG. 1.

FIG. 5 is an isometric view of the fluid transfer shield of FIG. 2 and a mold for forming the fluid transfer shield according to the present disclosure.

FIG. 6A is an illustration of a user holding the fluid transfer shield of FIG. 1 and a syringe.

FIG. 6B is an illustration of the user fluidly coupling the syringe of FIG. 6A with the fluid transfer shield of FIG. 1.

FIG. 7 is an illustration of the user fluidly coupling a secondary container with the fluid transfer shield and the syringe of FIG. 6B according to the present disclosure.

FIG. 8 is an illustration of the user fluidly coupling a secondary syringe with the fluid transfer shield and the syringe of FIG. 6B according to the present disclosure.

FIG. 9 is an illustration of the fluid transfer shield of FIG. 1 fluidly coupled to a secondary container that is seated in a container rack according to the present disclosure.

FIG. 10 is a side view of a fluid transfer shield according to the present disclosure.

FIG. 11 is an isometric view of a fluid transfer shield according to the present disclosure.

FIG. 12 is a top view of the fluid transfer shield of FIG. 11.

FIG. 13 is a side view of the fluid transfer shield of FIG. 11.

FIG. 14 is another side view of the fluid transfer shield of FIG. 11.

FIG. 15A is a schematic illustration of a syringe being attached to the fluid transfer shield of FIG. 11.

FIG. 15B is a schematic illustration of a secondary container fluidly coupled to the syringe and the fluid transfer shield of FIG. 15A.

FIG. 16 is a side view of a fluid transfer shield according to the present disclosure.

FIG. 17 is a side view of a fluid transfer shield according to the present disclosure.

FIG. 18 is a side view of a fluid transfer shield according to the present disclosure.

FIG. 19 is a side view of a fluid transfer shield according to the present disclosure.

FIG. 20 is a side view of a fluid transfer shield according to the present disclosure.

### DETAILED DESCRIPTION

It is to be understood that the present disclosure is not limited in its application to the details of construction and the arrangement of components set forth in the following description or illustrated in the following drawings. The systems and methods described herein are capable of other configurations and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Also as used herein, unless otherwise specified or limited, directional terms are presented only with regard to the particular configuration and perspective described. For example, reference to features or directions as “horizontal,” “vertical,” “front,” “rear,” “left,” “right,” and so on are generally made with reference to a particular figure or example and are not necessarily indicative of an absolute orientation or direction. However, relative directional terms for a particular configuration may generally apply to alternative orientations of that configuration. For example, “front” and “rear” directions or features (or “right” and “left” directions or features, and so on) may be generally understood to indicate relatively opposite directions or features.

As used herein in the context of activities or engagement of components, unless otherwise specified or limited, “manual” refers to the use of human hands. In some cases, “manual” engagement or activity can include direct manual engagement or activity: i.e., engagement or activity directly conducted by a user's hands (e.g., a user grasping or manipulating an object by hand). In some cases, “manual” engagement or activity can include engagement or activity via a non-powered hand tool (e.g., pliers).

The following discussion is presented to enable a person skilled in the art to make and use configurations of the present disclosure. Various modifications to the illustrated configurations will be readily apparent to those skilled in the art, and the generic principles herein can be applied to other configurations and applications without departing from configurations of the present disclosure. Thus, configurations of the disclosure are not intended to be limited to configurations shown, but are to be accorded the widest scope consistent with the principles and features disclosed herein. The following detailed description is to be read with reference to the figures, in which like elements in different figures have like reference numerals. The figures, which are not necessarily to scale, depict selected configurations and are not intended to limit the scope of configurations of the disclosure. Skilled artisans will recognize the examples provided herein have many useful alternatives and fall within the scope of configurations of the disclosure

As noted above, in some instances, it may be necessary to transfer fluid, such as blood, from a syringe to a secondary container. In the practice of phlebotomy where a relatively small amount of blood is taken from a patient (e.g., pediatric or elderly phlebotomy), the obtained blood does not pass through a closed transfer system. Instead, donor blood is often extracted via a syringe. The donor blood can then be transferred to a secondary container for testing or transport, such as a microtainer (e.g., BD Microtainer®) or a blood gas syringe. The transfer process from the donor syringe to the secondary container can expose the user (e.g., a healthcare worker) or the immediate environment to the blood via unwanted or unintentional blood spray. Other risks associated with the transfer process can include needle stick, which can increase the chances of blood borne pathogen exposure.

Configurations of the present disclosure can address these and other risks associated with blood transfer. Generally, configurations of the disclosure provide a fluid transfer shield. The fluid transfer shield can be configured as a blood transfer shield and can provide a fluid coupling between a first container and a second container. For example, a first container, such as a syringe (e.g., a donor syringe, a phlebotomy syringe, etc.) having blood contained therein can be coupled to a first port of the transfer shield and a secondary container, such as a microtainer, can be coupled to a second port of the transfer shield to fluidly couple the first container and the secondary container. The fluid transfer shield can allow blood from the first container to be safely deposited into the second container while generally reducing or containing unwanted blood spray.

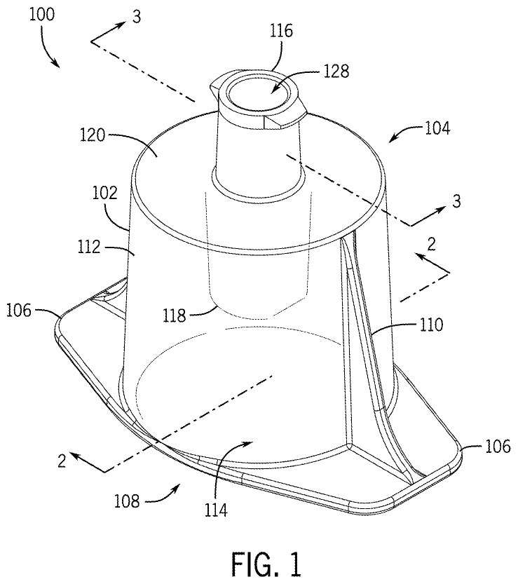

FIGS. 1-4 illustrate a fluid transfer shield 100 according to one configuration of the present disclosure. With reference to FIG. 1, the fluid transfer shield 100 includes a body 102 having a shield 104. The body 102 also includes first and second flanges 106 extending from a base 108 of the shield 104. The first and second flanges 106 are generally symmetric about a center line of the body 102 and extend from opposing sides of the shield 104. First and second support webs 110, which can be generally configured as buttresses, extend from an exterior 112 of the shield 104 to a respective one of the first and second flanges 106.

In the illustrated configuration, the shield 104 defines a generally cylindrical geometry. However, other geometries are possible, such as, for example, flared or conical. In this regard, the base 108 of the shield 104 is generally circular and the shield 104 defines an interior space 114 opposite the exterior 112. The body 102 further includes a first port 116 positioned and generally extending from the exterior 112 of the shield 104 and a second port 118 positioned and generally extending within the interior space 114 of the shield 104.

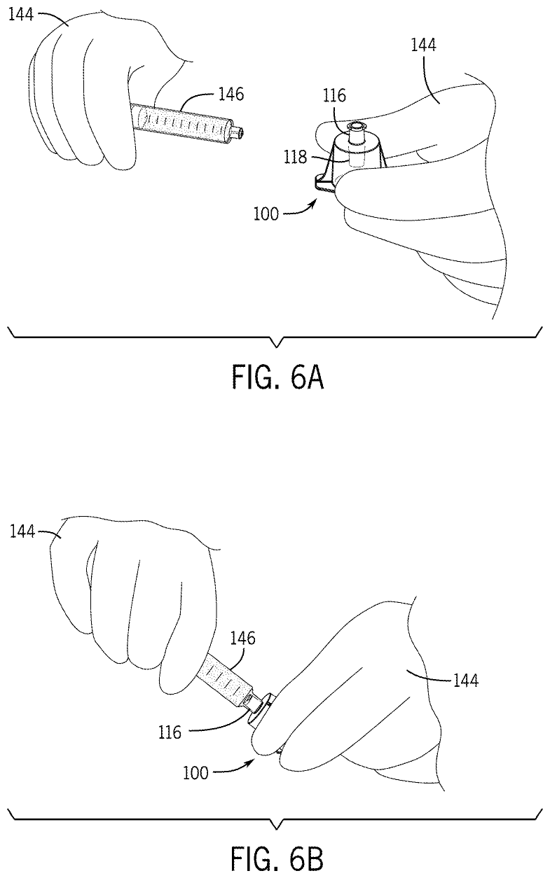

Also shown in FIG. 1, a footprint of the fluid transfer shield 100 formed by the bottom of the body 102 (with respect to the orientation illustrated in FIG. 1) defines an oblong geometry. The footprint generally includes the lower perimeter of the shield 104 and the lower perimeters of each of the first and second flanges 106. The oblong geometry can advantageously provide a comfortable and secure grip shape when employed by a user, as will be discussed in more detail below with reference to FIGS. 6A and 6B.

Opposite the base 108 of the shield 104, the first port 116 extends from a top 120 of the body 102. The first port 116 can be configured as a cylindrical projection with a tapered side wall. In some configurations, the first port 116 can include a Luer lock. In general, Luer locks can provide a standardized system of small-scale fluid fittings for making leak-free or leak-resistant connections between a male-taper fitting and a mating female part. Mating female Luer locks can often be found on medical laboratory instruments, including syringe tips (e.g., hypodermic syringe tips) needles, and stopcocks. In this regard, the first port 116 can include a female Luer lock connector configured to fluidly couple the first port 116 to a first container, such as a donor syringe. However, it should be appreciated that other connection types are possible, including geometries that provide a snap fit, an interference fit, a threaded connection, or other connections configured to provide a leak-free or leak-resistant coupling.

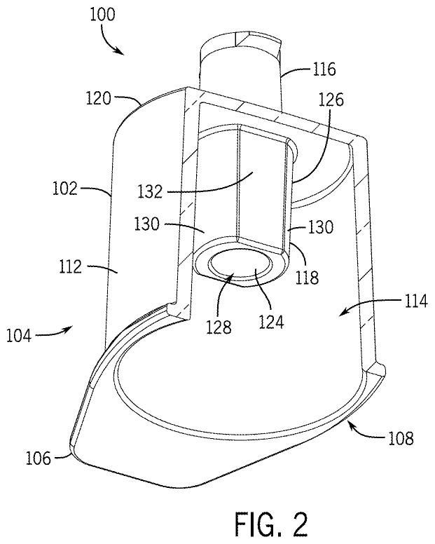

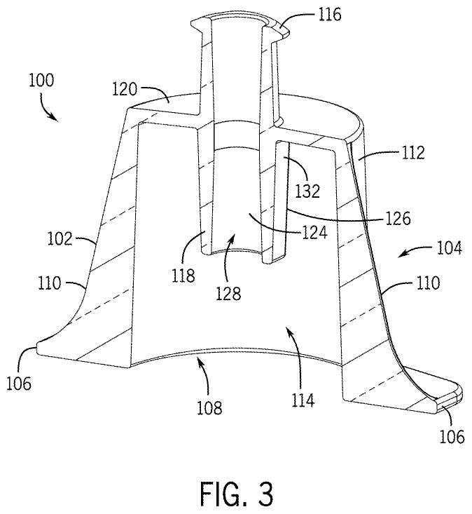

The second port 118 is fluidly coupled to the first port 116 and extends within the interior space 114 of the shield 104 opposite the first port 116. Notably, as further illustrated in FIGS. 2-4, the second port 118 extends only partially from the top 120 into the interior space 114 toward the base 108 (i.e., the second port 118 does not extend the full axial length of the shield 104). With reference to FIG. 2, the second port 118 includes an interior surface 124 and an exterior surface 126. The interior surface 124 defines a passageway 128 that extends through each of the first port 116 and the second port 118 (see, for example, FIG. 3)

With continued reference to FIG. 2, the passageway 128 can have a generally circular cross section. In contrast, the exterior surface 126 of the second port 118 does not have a circular cross section. In particular, the exterior surface 126 includes a first pair of faces 130 and a second pair of faces 132. Each of the first pair of faces 130 are opposite second port 118 from each other and the second pair of faces 132 are opposite the second port 118 from each other. In the illustrated configuration, the first pair of faces 130 are configured as curved surfaces that extend the length of the second port 118 in the axial direction and the second pair of faces 132 are configured as planar (e.g., flat) surfaces that extend the length of the second port 118 in the axial direction.

In use, a secondary container, such as a test tube or microtainer can be secured to the second port 118 to receive fluid, such as blood, from a syringe coupled to the first port 116 via the passageway 128. In particular, the secondary container can be fluidly coupled to the second port 118 so that the second port 118 extends into an opening of the secondary container (i.e., the exterior surface 126 can be inserted into the secondary container). When the second port 118 is fluidly coupled to the secondary container, the second pair of faces 132 can provide a relief path (e.g., an opening, a channel, etc.). The relief path can effectively release air from the secondary container during a fluid transfer process so that the fluid flows smoothly (e.g., without a vacuum or pressure interruption) from a syringe into the secondary container.

With reference to FIG. 3, the exterior surface 126 of the second port 118 can generally provide a tapered geometry. In particular, the second port 118 can be narrower at a distal end (i.e., the end facing the base 108) and wider at a proximal end (i.e., near the top 120 of the body 102). The tapered geometry can promote a leak-free or leak-resistant coupling as the fluid transfer shield 100 is coupled to the secondary container. As described above, the second port 118 extends within the interior space 114 of the shield 104 and is laterally surrounded by the shield 104. The shield 104 can surround the second port 118 in the lateral direction (i.e., a direction perpendicular to the axial direction defined by the fluid transfer shield 100) to catch, stop, and/or limit fluid spatter as fluid is transferred from a first container to a secondary container. In general, the shield 104 can act as an umbrella to capture or contain fluid, such as blood or other biofluids (e.g., urine, saliva, etc.), that may be otherwise hazardous or unsanitary if sprayed or released outside first or second containers between which the fluid is to be transferred.

In general, the fluid transfer shield 100 can define certain geometric ratios that advantageously promote usability and functionality. For example, certain height and width dimensions, particularly height and width ratios, can provide a user with comfortable and intuitive grip position and orientation such that a user may be able to quickly and efficiently couple the fluid transfer shield 100 to a donor syringe and a secondary container. In one example, a height and width ratio of the fluid transfer shield 100 can promote single-handed use, such that a user can actuate a plunger of a donor syringe (e.g., via a thumb) coupled to the fluid transfer shield while simultaneously gripping the fluid transfer shield 100 with the same hand (e.g., index and middle fingers at the first and second flanges 106). In some embodiments, the height and width ratio of the shield 104 may be 1:1 to promote a certain grip, such as, for example, a thumb and a first finger can secure the fluid transfer shield 100 to a first container at the first port 116 and a second finger can help secure a secondary container to the fluid transfer shield at the second port 118.

Other advantageous dimensional ratios of the fluid transfer shield 100 can include the diameter of the base 108 of the shield 104 and the height of the shield 104 in combination with the length that the second port 118 extends into the interior space 114 of the shield 104. These and other ratios, for example, can provide a sufficient shielding zone so that blood or other fluid is transferred into a secondary container without spraying onto or near the user, while the overall fluid transfer shield 100 maintains a size to comfortably fit in and average-sized adult hand. In this regard, a ratio of the height of the shield 104 to the length of the secondary container may be preferably between approximately 1:3 and 1:2 so that about one third to one half of the secondary container is laterally covered by the shield 104 in the axial direction when coupled to the second port 118. However, in other embodiments, such as when a fluid transfer shield similar to the fluid transfer shield 100 is used in the transfer of urine, for example, only 10% of the length of a secondary container may be laterally surrounded by the shield 104 to sufficiently transfer fluid between first and second containers.

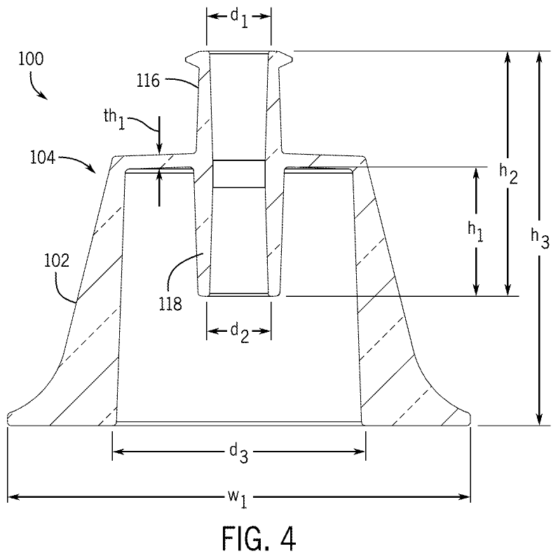

In this regard, FIG. 4, in combination with the table below, provides non-limiting examples of dimensions of the fluid transfer shield 100. It should be appreciated that the dimensions below are by way of example, and that other dimensions are possible. In particular, configurations can include dimensions that vary from the below values by approximately 20%, as just one example.

| h₁ (mm) | h₂ (mm) | h₃ (mm) | d₁ (mm) | d₂ (mm) | d₃ (mm) | th₁ (mm) | w₁ (mm) |
|---|---|---|---|---|---|---|---|
| 9.5 | 18 | 27.5 | 4.31 | 4.31 | 18 | 1 | 34 |

The dimensions above refer generally to different heights, widths, and diameters of the fluid transfer shield 100. For example, h₁ can correspond to the length that the second port 118 extends into the interior space 114 of the shield 104, h₂ can correspond to the length of the passageway 128, h₃ can correspond to the overall height of the fluid transfer shield 100, including the shield 104 and the first port 116, d₁ can correspond to the diameter of the passageway 128 at a distal end of the first port 116, d₂ can correspond to the diameter of the passageway 128 at a distal end of the second port 118, d₃ can correspond to the opening diameter of the shield 104 at the base 108, th₁ can correspond to the thickness of the body 102 at the top 120 of the fluid transfer shield 100, and w₁ can correspond to an over width of the body 102 extending between each of the flanges 106 (i.e., in the longer direction of the oblong footprint). It should be appreciated that additional dimensions of the fluid transfer shield 100 can be interpolated from the dimensions outlined in FIG. 4. For example, a body height h_{b} (not shown) of the shield 104 can be calculated from:

h_{b}=h₃−h₂+h₁+th₁

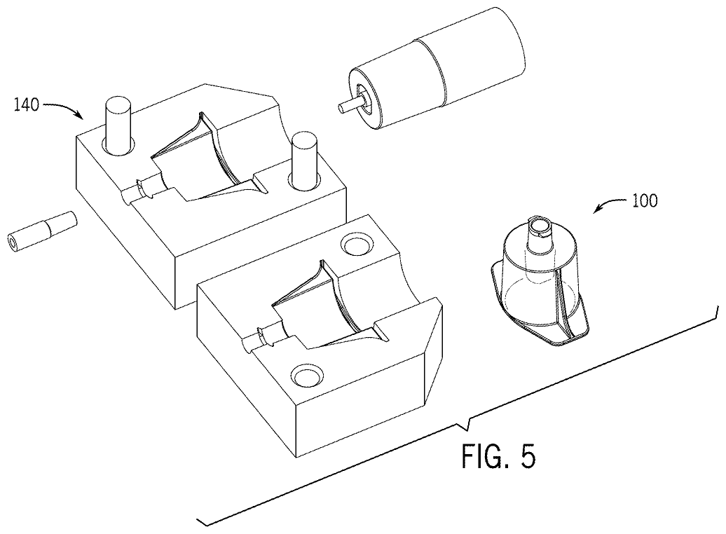

Referring now to FIG. 5, in one example, the fluid transfer shield 100 can be produced or manufactured via a molding process, such as injection molding, using a mold 140. In some configurations, the fluid transfer shield 100 can be molded using a translucent, near-translucent, or semi-translucent polymer, such as polypropylene, for example. However, in other configurations, other thermoplastics are possible. In some configurations, during a manufacturing process, the fluid transfer shield 100 can be packaged in a sterile blister pack. However, in other configurations, the fluid transfer shield 100 may not be packaged in a sterile pack. A user, such as a healthcare professional, can then open the pack to access the fluid transfer shield 100 to aid in the fluid transfer from a first container to a secondary container.

FIGS. 6A and 6B illustrate an exemplary user 144 fluidly coupling a first container, configured as a syringe 146, to the first port 116 of the fluid transfer shield 100. In the illustrated example, the syringe 146 contains blood that is to be transferred to a secondary container. As described above, the syringe 146 can be secured to the fluid transfer shield 100 via a Luer lock connection. In this regard, the user 144 may twist the syringe 146 relative to the fluid transfer shield 100 to secure the syringe 146 to the first port 116.

In use, the user 144 can grip the shield 104 of the fluid transfer shield 100 at the exterior 112 on either sides of the flanges 106 and support webs 110. The general oblong shape of the fluid transfer shield 100 near the base 108 of the shield 104 provides a grip geometry that can be conducive to securely coupling the syringe 146 and the first port 116. For example, the flanges 106 can provide a point of leverage when the user rotates the fluid transfer shield 100 relative to the syringe.

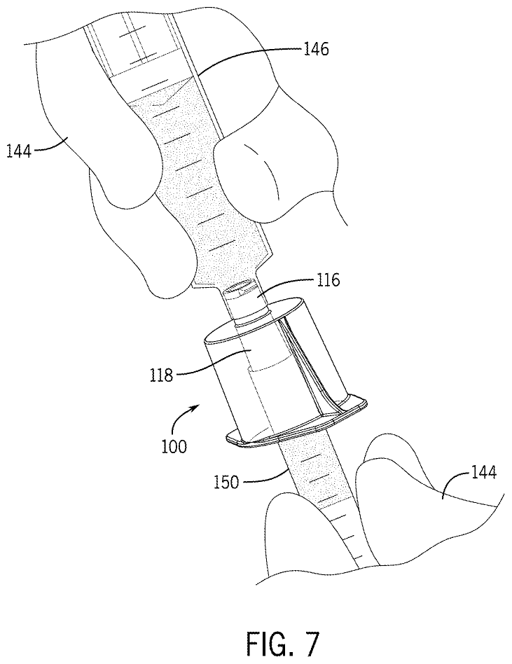

FIG. 7 illustrates one example of a secondary container configured as a microtainer 150 that can be coupled to the second port 118 of the fluid transfer shield 100. As shown in FIG. 7, the syringe 146 and the microtainer 150 are fluidly coupled via the passageway 128 extending between the first port 116 and the second port 118 of the fluid transfer shield 100. As blood is transferred from the syringe 146 to the microtainer 150, the shield 104 surrounds the microtainer 150 near the opening of the microtainer 150 to prevent or limit blood exposure outside of the syringe 146 and the microtainer.

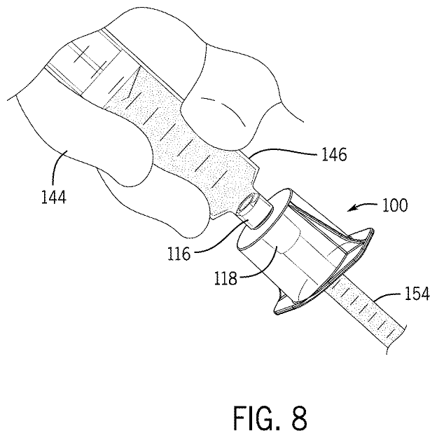

FIG. 8 illustrates another example of a secondary container configured as a second syringe 154 that can be coupled to the second port 118 of the fluid transfer shield 100. Like the example shown in FIG. 7, the syringe 146 and the second syringe 154 are fluidly coupled via the passageway 128 extending between the first port 116 and the second port 118 of the fluid transfer shield 100. In the illustrated embodiment, the second syringe 154 can be inserted into the second port 118 to fluidly couple the second syringe 154 and the passageway 128. As blood is transferred from the syringe 146 to the second syringe 154, the shield surrounds the second syringe 154 near the syringe tip of the second syringe 154 to prevent or limit blood exposure outside either of the syringes 146, 154.

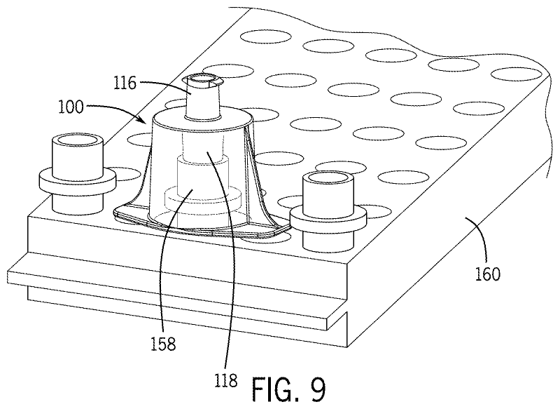

FIG. 9 illustrates another example of a secondary container configured as a collection tube 158 that can be coupled to the second port 118 of the fluid transfer shield 100. In the illustrated configuration, the collection tube 158 can be seated in a collection tube rack 160. The fluid transfer shield 100 can be advantageously dimensioned to engage the collection tube 158 without having to remove the collection tube 158 from the collection tube rack 160. This can expedite the safe filling of multiple collection tubes 158 from a single donor syringe, if necessary.

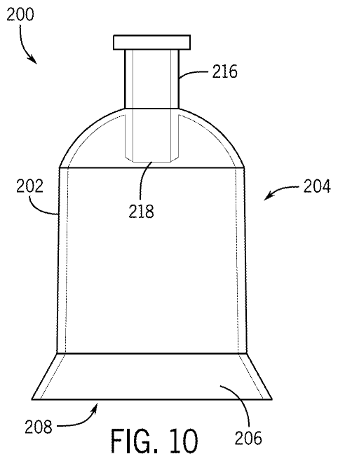

FIG. 10 illustrates another exemplary configuration of a fluid transfer shield 200 according to another configuration of the present disclosure. Similar to the fluid transfer shield 100, the fluid transfer shield 200 defines a body 202 having a shield 204 and a flange 206. In the illustrated configuration, the flange 206 is configured as a continuous annular flange that surrounds a base 208 of the shield 204. In use, the flange 206 can provide a grip for a user to grasp when securing a first container to a first port 216 of the fluid transfer shield 200 and/or a secondary container to a second port 218 of the fluid transfer shield 200.

Like the fluid transfer shield 100, the shield 204 of the fluid transfer shield 200 laterally surrounds the second port 218, which extends into an interior space of the shield opposite the first port 216. The fluid transfer shield 200 can be operated in substantially the same manner as the fluid transfer shield 100, and can be used with first and second containers similarly to those illustrated in FIGS. 6-9.

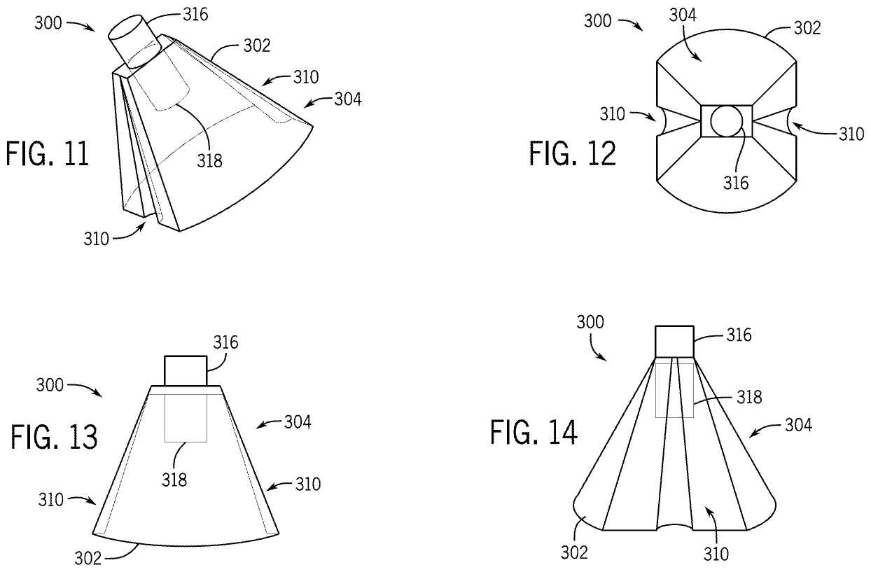

FIGS. 11-14 illustrate another exemplary configuration of a fluid transfer shield 300 according to another configuration of the present disclosure. Similar to the fluid transfer shields described above, the fluid transfer shield 300 defines a body 302 having a shield 304. In the illustrated configuration, the shield 304 defines grip areas 310 on opposing sides of the shield 304. In use, the grip areas 310 can provide a grip for a user to grasp when securing a first container to a first port 316 of the fluid transfer shield 300 and/or a secondary container to a second port 318 of the fluid transfer shield 300. In some embodiments, grip areas can include ribbing or other exterior texture to enhance a user's grip when using a transfer shield.

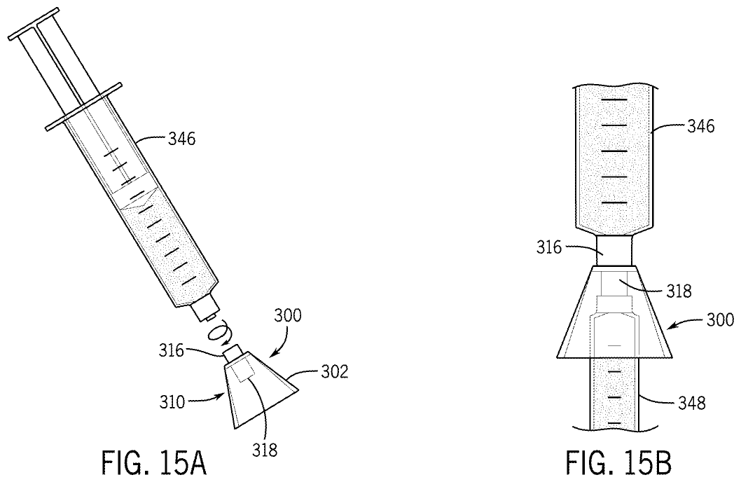

With reference to FIGS. 15A and 15B, in use, a syringe 346 can be fluidly coupled to the fluid transfer shield 300 at the first port 316 by twisting one of the syringe 346 or the fluid transfer shield 300 relative to the other. A secondary container 348 can then be secured to the fluid transfer shield 300 at the second port 318 to fluidly couple the syringe 346 with the secondary container 348. Notably, when the secondary container 348 is secured to the second port 318, the shield 304 laterally surrounds the secondary container 348 near the opening of the secondary container 348.

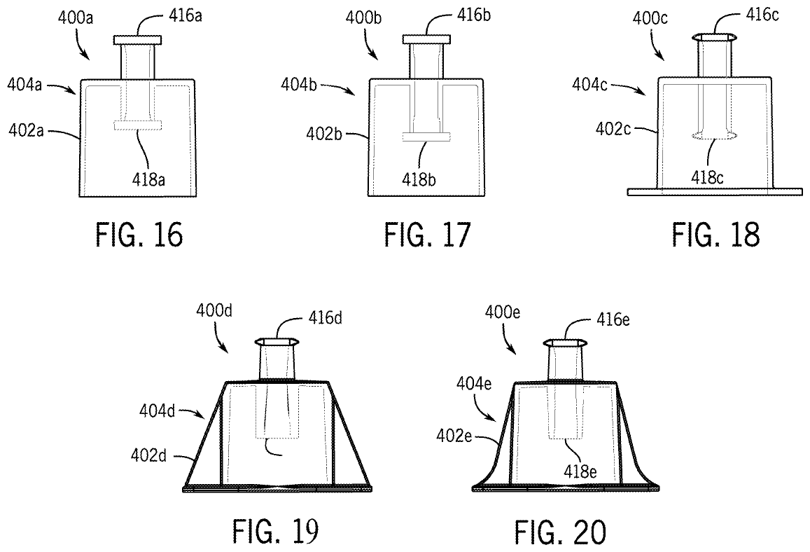

FIG. 16-20 illustrate additional exemplary configurations of blood transfer shields 400a-e. Similar to the blood transfer shields described above, each of the blood transfer shields 400a-e defines a respective body 402a-e having a shield 404a-e. A respective first port 416a-e extends from a top of the body 402a-e outside of the shield 404a-e and a respective second port 418a-e extends opposite the first port 416a-e within an interior space of the shield 404a-e so that the shield laterally surrounds the second port 418a-e. In some embodiments, such as illustrated in FIGS. 16-18, the respective second ports 418a-c can include a Luer lock, which may be used with a secondary container having a corresponding mating Luer lock. In other embodiments, second ports, such as second ports 418a-c can include one or more flanges extending therefrom to secure a secondary container to the blood transfer shield.

Further illustrated in each of FIGS. 18-20, the fluid transfer shields 400c-e can include a respective flange 406c-e adjacent to a base of the shield 404c-e. The flanges 406c-e can provide a grip for a user to grasp when securing a first container to the first port 416c-e and/or a secondary container to the second port 418c-e. In some embodiments, blood transfer shields, such as the blood transfer shields 400c-e can include first, second, third, and fourth flanges radially extending and spaced around the base of the shield to provide a secure and non-orientation specific grip. In general, the fluid transfer shields 400a-e can be operated in substantially the same manner as the fluid transfer shields described above, and can be used with first and second containers similarly to those illustrated in FIGS. 6-9.

Thus, while the invention has been described above in connection with particular configurations and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

We claim:

1. A fluid transfer shield to facilitate transferring fluid from a first container to a secondary container, the fluid transfer shield comprising:
a shield having an exterior and defining an interior space;
a first port extending from the exterior of the shield, the first port configured to be fluidly coupled to the first container;
a second port extending opposite the first port and into the interior space of the shield so that the shield laterally surrounds the second port, the second port configured to be fluidly coupled to the secondary container; and
a passageway extending between the first port and the second port to fluidly couple the first container and the secondary container when the first container is fluidly coupled to the first port and the secondary container is fluidly coupled to the second port.

2. The fluid transfer shield of claim 1, wherein the second port includes a relief path to allow airflow from the interior space to or from at least one of the passage or the second port.

3. The fluid transfer shield of claim 2, wherein the relief path is formed by a pair of planar faces on opposing sides of the second port, the pair of planar faces configured to form a channel between an exterior surface of the second port and an interior surface of the secondary container when the secondary container is engaged with the second port.

4. The fluid transfer shield of claim 1, further comprising a flange extending radially from the exterior of the shield proximate to a base of the shield.

5. The fluid transfer shield of claim 4, wherein first and second support ribs extend from the exterior of the shield to the flange to rigidly support the flange.

6. The fluid transfer shield of claim 4, wherein a perimeter of fluid transfer shield that includes the shield and the flange adjacent to the base of the shield forms an oblong footprint.

7. The fluid transfer shield of claim 1, wherein the second port includes an interior surface that defines the passageway and an exterior surface, the exterior surface having a tapered geometry so that a distal end of the second port is narrower than a proximal end of the second port.

8. The fluid transfer shield of claim 1, wherein the first port includes a female Luer lock connector configured to receive a syringe having a male Luer lock connector as the first container to fluidly couple the syringe to the first port via the female Luer lock connector.

9. The fluid transfer shield of claim 1, wherein the second port extends between approximately 7 millimeters and 11 millimeters into the interior space of the shield and the shield defines a body height between approximately 16 millimeters and 24 millimeters.

10. The fluid transfer shield of claim 1, wherein an opening diameter of the shield is between approximately 14 millimeters and 22 millimeters and an overall height of the fluid transfer shield in an axial direction is between approximately 22 millimeters and 33 millimeters.

11. A blood transfer shield for fluidly coupling a syringe and a secondary container, the blood transfer shield comprising:
a shield having a cylindrical body and a base that defines an opening at a first end of the cylindrical body;
a passageway extending through the cylindrical body of the shield, the passageway formed at a second end of the cylindrical body opposite the base; and
a first port that forms a first end of the passageway, the first port configured to securely engage the syringe to fluidly couple the syringe to the passageway.

12. The blood transfer shield of claim 11, further comprising a second port that forms a second end of the passageway, the second port laterally surrounded by the shield.

13. The blood transfer shield of claim 12, wherein the second port defines an exterior surface opposite the passageway, the exterior surface configured to be inserted into an opening of a secondary container to fluidly couple the secondary container with the passageway.

14. The blood transfer shield of claim 13, wherein the exterior surface includes a first pair of faces and a second pair of faces, the first pair of faces configured as curved surfaces that extend along the second port in an axial direction and the second pair of faces configured as flat surfaces that extend along the second port in the axial direction.

15. The blood transfer shield of claim 14, wherein the second pair of faces are configured to form an air relief channel with an opening of the secondary container when the secondary container is fluidly coupled with the passageway.

16. The blood transfer shield of claim 11, further comprising:
a flange extending radially from the cylindrical body of the shield adjacent to the opening; and
first and second buttresses extending downward from the second end of the cylindrical body to the flange.

17. The blood transfer shield of claim 11, wherein the shield comprises a semi-translucent polymer.

18. The blood transfer shield of claim 12, wherein the first port and the second port are each tapered so that a respective distal end is narrower than a respective proximal end of the first and second ports.

19. A method of transferring blood from a first container to a secondary container, the method comprising:
drawing blood into the first container;
fluidly coupling the first container to a first port of a blood transfer shield, the first port extending from an exterior surface of the blood transfer shield;
fluidly coupling the secondary container to a second port of the blood transfer shield, the second port extending within an interior space defined by a lateral side wall of a shield of the blood transfer shield; and
expelling blood from the first container into the secondary container via a passageway extending between the first port and the second port.

20. The method of claim 19, wherein fluidly coupling the first container to the secondary container includes twisting the first container relative to the blood transfer shield.
